(19) United States
(12) Patent Application Publication
(10) Pub. No.: US 2023/0296628 A1
(43) Pub. Date: Sep. 21, 2023

(54) Biomarkers for Identifying Relapses in Multiple Sclerosis

(71) Applicants: Oklahoma Medical Research Foundation, Oklahoma City, OK (US); The Board of Regents of the University of Oklahoma, Norman, OK (US)

(72) Inventors: Robert Axtell, Oklahoma City, OK (US); Kumar Gaurav, Oklahoma City, OK (US); Agnieshka Agasing, Orlando, FL (US)

(21) Appl. No.: 18/004,479

(22) PCT Filed: Jul. 8, 2021
(86) PCT No.: PCT/US2021/040912

§ 371 Date: Jan. 6, 2023

Related U.S. Application Data
(60) Provisional application No. 63/049,859, filed on Jul. 9, 2020.

(51) Int. Cl. G01N 33/68; G01N 33/569

(52) U.S. Cl. CPC G01N 33/6896; G01N 33/56972; G01N 2333/545; G01N 2333/70596; G01N 2800/285; G01N 2800/52; G01N 2800/54

(57) ABSTRACT

The present invention includes a method of predicting and/or treating a recurrence of MS treating a patient with multiple sclerosis, the method comprising: obtaining a hematopoietic cell sample from a patient suspected of having a recurrence of multiple sclerosis (MS), wherein the patient was in relapse for MS; determining the number of CD19⁺, CD24⁺, CD38⁺ transitional B cells in the hematopoietic cell sample, and a level of expression of neurofilament light (NFL) and interleukin-1β (IL-1β), which is predictive of a recurrence of MS; and treating the MS patient with recurrence until there is an increase in CD19⁺, CD24⁺, CD38⁺ transitional B cells and/or a decrease in a level of expression of NFL and interleukin-1β (IL-1β) when compared to an untreated MS control sample, an unresponsive MS control sample, or an MS patient with long-term stable disease sample.

### CROSS-REFERENCE TO RELATED APPLICATIONS

This PCT International Application claims benefit to U.S. Provisional Application No. 63/049,859 filed on Jul. 9, 2020, the contents of which are incorporated by reference in their entirety.

### STATEMENT OF FEDERALLY FUNDED RESEARCH

This invention was made with government support under R01AI137047 and R01EY027346 awarded by the National Institutes of Health. The government has certain rights in the invention.

### TECHNICAL FIELD OF THE INVENTION

The present invention relates in general to the field of biomarkers for multiple sclerosis, and more particularly, to the identification and treatment of patients with the relapse of multiple sclerosis.

### BACKGROUND OF THE INVENTION

Without limiting the scope of the invention, its background is described in connection with of multiple sclerosis.

In the work of Kuhle, et al., “Blood neurofilament light chain as a biomarker of MS disease activity and treatment response”, Neurology, 2019;92:e1007-e1015, the authors assess the value of blood neurofilament light chain (NfL) as a biomarker of recent, ongoing, and future disease activity and tissue damage and its utility to monitor treatment response in relapsing-remitting multiple sclerosis. They conclude that Blood NfL levels are associated with clinical and MRI-related measures of disease activity and neuroaxonal damage and have prognostic value.

One such patent is U.S. Patent Publication No. 2016/0054320, filed by Schubert, et al., is entitled “Markers For Determination Of Patient Responsiveness”, in which the applicants are said to teach compositions and methods for classification of individuals suffering from a demyelinating disease into groups that are informative of the individual's responsiveness or lack of responsiveness to treatment with a J3-interferon (IFNJ3) acting therapy. In particular, it is said that the effective immunomodulatory treatment of demyelinating disease with IFNJ3 is associated with an increase in circulating transitional B cells in the patient.

However, despite these advances, what is needed is a non-invasive and cost-effective biomarkers to monitor disease in multiple sclerosis (MS) patients, and to therapeutically intervene upon an indication that there may be a recurrence of MS.

### SUMMARY OF THE INVENTION

In one embodiment, the present invention includes a method of treating a patient with multiple sclerosis, the method comprising: obtaining a hematopoietic cell sample from a patient having multiple sclerosis (MS), wherein the patient was in relapse for MS; determining the number of CD19⁺, CD24⁺, CD38⁺ transitional B cells in the hematopoietic cell sample, and a level of expression of neurofilament light (NFL) and an inflammatory marker; and treating the MS patient with a therapeutic agent selected from at least one of: monomethyl fumarate, ozanimod, diroximel fumarate, cladribine, siponimod, ocrelizumab, daclizumab, alemtuzumab, PEG-interferon beta-1a, dimethyl fumarate, teriflunomide, fingolimod, natalizumab, laquinimod, ublituximab, or ponesimod, when the patient has a decreased number of CD19⁺, CD24⁺, CD38⁺ transitional B cells, and an increase in NFL inflammatory marker relative to an untreated MS control sample, an unresponsive MS control sample, or an long-term stable disease sample. In one aspect, the inflammatory marker is interleukin-1β (IL-1β). In another aspect, the determining step for CD19⁺, CD24⁺, CD38⁺ transitional B cells comprises: contacting the hematopoietic cell sample with reagents that detect CD19, CD24 and CD38 as transitional B cell markers; and quantitating those cells that are positive for expression of at least one of CD19, CD24 and CD38, when compared to a normal sample, or a sample from the untreated MS control sample, the unresponsive MS control sample, or the long-term stable disease sample. In another aspect, the determining step is performed by flow cytometry. In another aspect, the patient is human.

In another embodiment, the present invention includes a method of treating a multiple sclerosis patient, the method comprising: treating the MS patient with a therapeutic agent; obtaining a hematopoietic cell sample from the patient; determining the number of CD19⁺, CD24⁺, CD38⁺ transitional B cells in the hematopoietic cell sample, and a level of expression of neurofilament light (NFL) and interleukin-1β (IL-1β); and continuing treatment of the MS patient with the therapeutic agent for MS when the patient has a decreased number of CD19⁺, CD24⁺, CD38⁺ transitional B cells and an increase in the expression of NFL and IL-1β relative to an untreated or unresponsive MS control sample or a patient with long-term stable MS, wherein the patient is determined to be responsive to the therapeutic agent. In one aspect, the determining step for CD19⁺, CD24⁺, CD38⁺ transitional B cells comprises: contacting the hematopoietic cell sample with reagents that detect CD19, CD24 and CD38 as transitional B cell markers; and quantitating those cells that are positive for expression of at least one of CD19, CD24 and CD38, when compared to a normal sample, or a sample from the untreated MS control sample, the unresponsive MS control sample, or the long-term stable disease sample. In another aspect, the determining step is performed by flow cytometry. In another aspect, the patient is human. In another aspect, the therapeutic agent is selected from at least one of: monomethyl fumarate, ozanimod, diroximel fumarate, cladribine, siponimod, ocrelizumab, daclizumab, alemtuzumab, PEG-interferon beta-1a, dimethyl fumarate, teriflunomide, fingolimod, natalizumab, laquinimod, ublituximab, or ponesimod.

In another embodiment, the present invention includes a method of predicting and treating a recurrence of MS treating a patient with multiple sclerosis, the method comprising: obtaining a hematopoietic cell sample from a patient suspected of having a recurrence of multiple sclerosis (MS), wherein the patient was in relapse for MS; determining the number of CD19⁺, CD24⁺, CD38⁺ transitional B cells in the hematopoietic cell sample, and a level of expression of neurofilament light (NFL) and interleukin-1β (IL-1β), which is predictive of a recurrence of MS; and treating the MS patient with a therapeutic agent selected from at least one of: monomethyl fumarate, ozanimod, diroximel fumarate, cladribine, siponimod, ocrelizumab, daclizumab, alemtuzumab, PEG-interferon beta-1a, dimethyl fumarate, teriflunomide, fingolimod, natalizumab, laquinimod, ublituximab, or ponesimod when the patient has a decreased number of CD19⁺, CD24⁺, CD38⁺ transitional B cells, and an increase in NFL and inflammatory marker relative to the untreated MS control sample, the unresponsive MS control sample, or the long-term stable disease sample. In one aspect, the determining step for CD19⁺, CD24⁺, CD38⁺ transitional B cells comprises: contacting the hematopoietic cell sample with reagents that detect CD19, CD24 and CD38 as transitional B cell markers; and quantitating those cells that are positive for expression of at least one of CD19, CD24 and CD38, when compared to a normal sample, or a sample from the untreated MS control sample, the unresponsive MS control sample, or the long-term stable disease sample. In another aspect, the CD19⁺, CD24⁺, CD38⁺ cells are detected by flow cytometry. In another aspect, the patient is human.

In another embodiment, the present invention includes a method of predicting a recurrence of MS treating a patient with multiple sclerosis, the method comprising: obtaining a hematopoietic cell sample from a patient suspected of having a recurrence of multiple sclerosis (MS), wherein the patient was in relapse for MS; and determining the number of CD19⁺, CD24⁺, CD38⁺ transitional B cells in the hematopoietic cell sample, and a level of expression of neurofilament light (NFL) and interleukin-1β (IL-1β), which is predictive of a recurrence of MS; wherein an increase in CD19⁺, CD24⁺, CD38⁺ transitional B cells and/or a decrease in a level of expression of NFL and interleukin-1β (IL-1β) when compared to an untreated MS control sample, an unresponsive MS control sample, or an MS patient with long-term stable disease sample is indicative of a recurrence of MS. In one aspect, the individual is undergoing treatment with an at least one of: monomethyl fumarate, ozanimod, diroximel fumarate, cladribine, siponimod, ocrelizumab, daclizumab, alemtuzumab, PEG-interferon beta-1a, dimethyl fumarate, teriflunomide, fingolimod, natalizumab, laquinimod, ublituximab, or ponesimod. In another aspect, the determining step for CD19⁺, CD24⁺, CD38⁺ transitional B cells comprises: contacting the hematopoietic cell sample with reagents that detect CD19, CD24 and CD38 as transitional B cell markers; and quantitating those cells that are positive for expression of at least one of CD19, CD24 and CD38, when compared to a normal sample, or a sample from the untreated MS control sample, the unresponsive MS control sample, or the long-term stable disease sample. In one aspect, the CD19⁺, CD24⁺, CD38⁺ cells are detected by flow cytometry. In another aspect, the patient is human. In another aspect, the method further comprises the step of treating the patient with at least one of: monomethyl fumarate, ozanimod, diroximel fumarate, cladribine, siponimod, ocrelizumab, daclizumab, alemtuzumab, PEG-interferon beta-1a, dimethyl fumarate, teriflunomide, fingolimod, natalizumab, laquinimod, ublituximab, or ponesimod.

### BRIEF DESCRIPTION OF THE DRAWINGS

For a more complete understanding of the features and advantages of the present invention, reference is now made to the detailed description of the invention along with the accompanying figures and in which:

FIGS. 1A to 1F show that decreased transitional B cells and increased NFL and IL-1β levels are associated with active relapse in MS patients. Blood from DMT naïve MS patients experiencing either an active relapse, who recently recovered from a relapse or had long-term stable disease were analyzed for the following: (FIG. 1A) Transitional, naïve, class switched (CS)-memory and plasmablast B cell subsets using flow cytometry; (FIG. 1B) FACS plots representing the frequency of transitional B cells gated on CD19+B cells; (FIG. 1C) serum NFL levels; (FIG. 1D) correlation between transitional B cells and NFL levels; (FIG. 1E) IL-1β serum levels; and (FIG. 1F) correlation between transitional B cells and IL1β. Error bars represent SEM and ANOVA tests were used to determine statistical differences. Pearson correlation coefficient tests were used to measure linear correlations. P<0.05 was considered to be statistically significant.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

There is a growing need for non-invasive and cost-effective biomarkers to monitor disease in multiple sclerosis (MS) patients. Since neuro-inflammation and neurodegeneration are key characteristics of MS, markers associated with neurological damage and inflammation show considerable promise as biomarkers of disease activity.

Additionally, B cells are known to have multifunctional roles in MS pathology and different MS treatments have been shown to alter the composition of B cell subsets. Transitional B cells, for instance, have regulatory functions and have been associated with treatment efficacy while class switched (CS) memory B cells promote disease pathology in MS. Plasma cells, on the other hand, have been described to play both anti-inflammatory and pro-inflammatory roles in MS. The inventors disclose herein how to assesses different B cell subsets, neuronal markers such as neurofilament light (NFL) and inflammatory marker such as in interleukin-1β (IL-1β) that correlate with disease activity in MS.

The present invention uses three biomarkers to determine disease activity in multiple sclerosis patients: Neurofilament light (NFL), Interleukin-1B (IL-1B) and transitional B cells. High levels of NFL have been shown to be associated with MS relapses. However, high levels of NFL are not MS specific as other diseases with neuronal damage also have high NFL. The present invention uses IL-1B and transitional B cells in combination with NFL to be indicative of MS-specific neuronal damage, and more particularly, to be predictive and to determine a recurrence of MS, and can also be used to determine a treatment regimen, and to track disease progression and/or remission. When a relapse is detected, the patient can be treated with, for example, at least one of: monomethyl fumarate, ozanimod, diroximel fumarate, cladribine, siponimod, ocrelizumab, daclizumab, alemtuzumab, PEG-interferon beta-1a, dimethyl fumarate, teriflunomide, fingolimod, natalizumab, laquinimod, ublituximab, or ponesimod. The present invention can also be used with future treatments for MS.

The discovery of biomarkers that monitor disease processes in multiple sclerosis (MS) is an active area of research. Here, the present inventors determined if different B cell subsets in the blood correlate with active relapses in MS patients. It was found that, surprisingly, patients undergoing an active relapse had lower frequencies of the anti-inflammatory transitional B cell subset. Furthermore, the inventors found a negative correlation with transitional B cells and serum levels of neurofilament light (NFL) and interleukin-1β (IL-1β). This study demonstrates that monitoring B cell subsets along with NFL and IL-1β can be used to track disease activity in MS patients.

Multiple sclerosis (MS) is characterized by various symptoms and signs of CNS dysfunction, with remissions and recurring exacerbations. The most common presenting symptoms for MS are paresthesias in one or more extremities, in the trunk, or on one side of the face; weakness or clumsiness of a leg or hand; or visual disturbances, e.g., partial blindness and pain in one eye (retrobulbar optic neuritis), dimness of vision, or scotomas. Other common early symptoms are ocular palsy resulting in double vision (diplopia), transient weakness of one or more extremities, slight stiffness or unusual fatigability of a limb, minor gait disturbances, difficulty with bladder control, vertigo, and mild emotional disturbances; all indicate scattered CNS involvement and often occur months or years before the disease is recognized. Excess heat may accentuate symptoms and signs.

The course of MS disease is highly varied, unpredictable, and, in most patients, remittent. At first, months or years of remission may separate episodes, especially when the disease begins with retrobulbar optic neuritis. However, some patients have frequent attacks and are rapidly incapacitated; for a few the course can be rapidly progressive (primary progressive MS, PPMS). Relapsing remitting MS (RRMS) is characterized clinically by relapses and remissions that occur over months to years, with partial or full recovery of neurological deficits between attacks. Such patients manifest approximately 1 attack, or relapse, pre year. Over 10 to 20 years, approximately 50% of RRMS patients develop secondary progressive MS (SPMS) which is characterized by incomplete recovery between attacks and accumulation of neurologic deficits resulting in increasing disability.

MS Diagnosis is indirect, by deduction from clinical, radiographic (brain plaques on magnetic resonance [MR] scan), and to a lesser extent laboratory (oligoclonal bands on CSF analysis) features. Typical cases can usually be diagnosed confidently on clinical grounds. The diagnosis can be suspected after a first attack. Later, a history of remissions and exacerbations and clinical evidence of CNS lesions disseminated in more than one area are highly suggestive.

As used herein, the terms “subject” or “patient” refer to a mammal. Mammals other than humans can be advantageously used as subjects that represent animal models of MS. A subject can be male or female.

As used herein, the term “analyze” refers to determining a set of values associated with a sample by measurement of a marker (such as, e.g., presence or absence of a marker or constituent expression levels) in the sample and comparing the measurement against measurement in a sample or set of samples from the same subject or other control subject(s). The markers of the present teachings can be analyzed by any of various conventional methods known in the art. To “analyze” can include performing a statistical analysis to, e.g., determine whether a subject is a responder or a non-responder to a therapy (e.g., an IFN treatment as described herein).

As used herein, the term “sample” refers to any biological sample that is isolated from a subject, generally a sample that comprises leukocytes. A sample can include, without limitation, an aliquot of body fluid, whole blood, white blood cells or leucocytes, synovial fluid, lymphatic fluid, cerebrospinal fluid, bone marrow, ascites fluid, and interstitial or extracellular fluid. The term “sample” also encompasses the fluid in spaces between cells, including gingival crevicular fluid, bone marrow, cerebrospinal fluid (CSF), saliva, mucous, sputum, semen, sweat, urine, or any other bodily fluids. “Blood sample” can refer to whole blood or a fraction thereof, particularly peripheral blood mononuclear cells (PBMC), i.e. white blood cells or leucocytes. Samples can be obtained from a subject by any convenient means, as is known in the art.

As used herein, the term “dataset” refers to a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring, cell counts by microscopy, flow cytometry, and the like. The phrase also encompasses receiving a set of data, e.g., from a third party that has processed the sample to experimentally determine the dataset. Additionally, the phrase encompasses mining data from at least one database or at least one publication or a combination of databases and publications.

As used herein, the terms “measuring” or “measurement” refers to determining the presence, absence, quantity, amount, or effective amount of a substance in a clinical or subject-derived sample, including the presence, absence, or concentration levels of such substances, and/or evaluating the values or categorization of a subject's clinical parameters based on a control.

As used herein, the term “transitional B-cells” refers to immature B-cells, which can be distinguished from mature B-cells by variations in cell surface markers, antigenic specificity, relative responsiveness to B-cell receptor signals, TLR receptor signaling, and other coreceptors, as well as cytokine production. Unique combinations of markers allow discrimination of different stages of development. The most immature transitional B-cells and recent emigrants from the bone marrow are called T1 B-cells (transitional type 1 B-cells or T1 cells). Others are T2 B-cells (transitional type 2 or T2 cells).

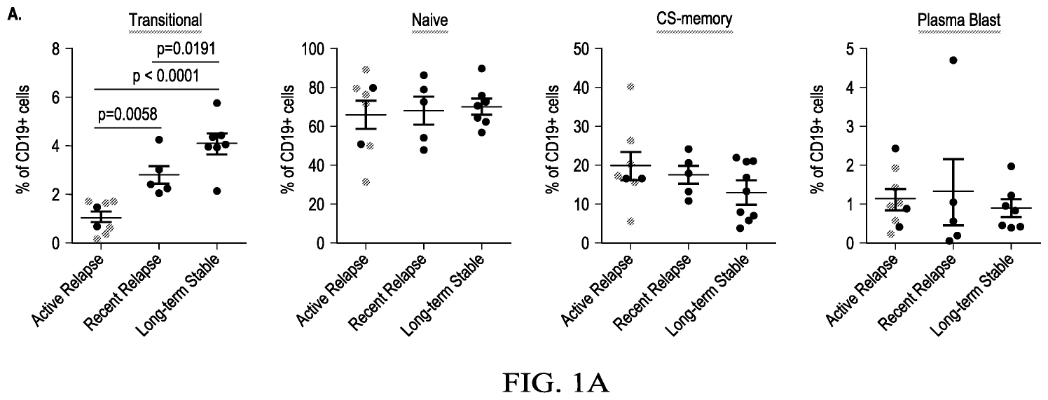

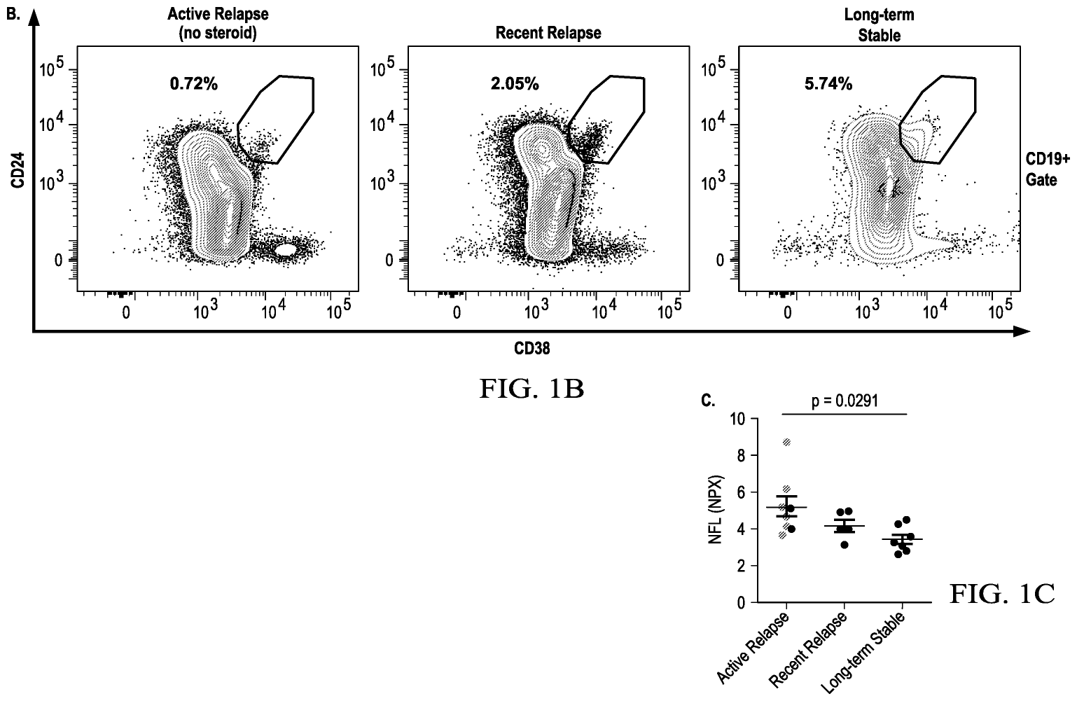

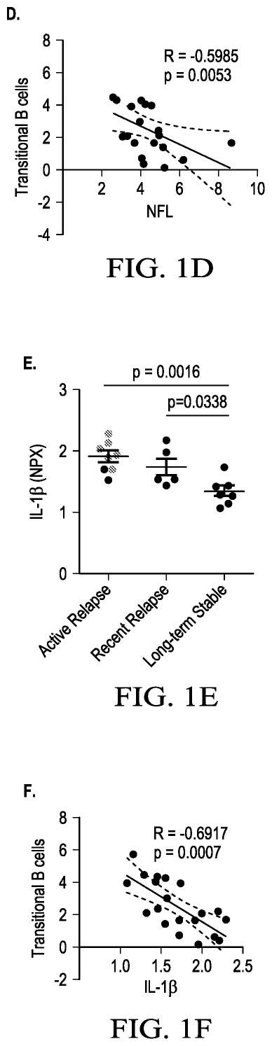

The number of transitional B cells may be read out as an absolute value, e.g. the number of transitional B cells per unit of blood, bone marrow, etc., where an increase in absolute number relative to an untreated or unresponsive control is determined. Conveniently, the transitional B cell count can be determined as a percentage of total B cells, or as a percentage of total lymphocytes. The number of transitional B cells as a percent of total B cells, e.g., as a percent of CD19⁺, CD24⁺, CD38⁺ cells from healthy control peripheral blood is usually less than about 3%. As shown in FIG. 1B, the percent CD19⁺, CD24⁺, CD38⁺ cells for active relapse patients is around 0.72%, while the percentage for MS patients in recent relapse is about 2.05%, compared to the percentage of cells for long-term stable MS patients being about 5.74%. The percentage of transitional B cells as a percent of total B cells, e.g. as a percent of CD19⁺, CD24⁺, CD38⁺ cells from, e.g., individuals responsive to IFN-b. Treatment is usually greater than about 3%, greater than about 5%, greater than about 6%, greater than about 7%, and may be higher, e.g. greater than about 10%, than about 12%, than about 15%.

The detection reagents can be provided as part of a kit, e.g., those to detect CD19⁺, CD24⁺, CD38⁺ on B cells to detect a decrease in transitional B cells, as well as to detect a level of expression of neurofilament light (NFL) and an inflammatory marker, such as, interleukin-1β (IL-1β). Thus, the invention further provides kits for detecting the presence of a panel of specific markers to assess the number of transitional B cells in a biological sample. Procedures using these kits can be performed by clinical laboratories, experimental laboratories, medical practitioners, or private individuals. The kits of the invention for detecting markers comprise affinity reagents useful for identifying transitional B cells, which can be provided in solution or bound to a substrate. The kit can optionally provide additional components that are useful in the procedure, including, but not limited to, buffers, developing reagents, labels, reacting surfaces, means for detection, control samples, standards, instructions, and interpretive information.

In addition to the above components, the subject kits will further include instructions for practicing the subject methods. These instructions can be present in the subject kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the interne to access the information at a removed site. Any convenient means can be present in the kits.

For a determination of recurrence of MS, the predictive ability of a model can be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about at least about 0.7, at least about 0.75, at least about 0.8, at least about 0.85, at least about 0.9, or higher.

General methods in molecular and cellular biochemistry can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), relevant portions incorporated herein by reference. Reagents, cloning vectors, and kits for genetic manipulation referred to in this disclosure are available from commercial vendors such as BioRad, Stratagene, Invitrogen, Sigma-Aldrich, and ClonTech, relevant portions incorporated herein by reference.

The invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. Due to biological functional equivalency considerations, changes can be made in protein structure without affecting the biological action in kind or amount. All such modifications are intended to be included within the scope of the appended claims.

As used herein, the term “treating” refers to both prevention of relapses, and treatment of pre-existing conditions. For example, the prevention of inflammatory disease can be accomplished by administration of the agent prior to development of a relapse. The treatment of ongoing disease, where the treatment stabilizes or improves the clinical symptoms of the patient, is of particular interest. The subject methods are used for prophylactic or therapeutic purposes.

Treatments for MS include interferon-b (Avonex, Betaseron, Rebif), Copaxone (Glatiramer acetate), and anti-VLA4 (Tysabri, natalizumab), which reduce relapse rate and to date have only exhibited a modest impact on disease progression. MS is also treated with immunosuppressive agents including methylprednisolone, other steroids, methotrexate, cladribine and cyclophosphamide. Many biological agents, such as anti-IFNg antibody, CTLA4-Ig (Abetacept), anti-CD20 (Rituxan), and other anti-cytokine agents in clinical development for MS.

Patient recruitment. RRMS patients were recruited and provided consent at the Oklahoma Multiple Sclerosis Center of Excellence under Institutional Review Board—approved protocols. Patients were diagnosed using the revised McDonald criteria (58) and disease activity was assessed by credentialed neurologists and confirmed with MRI.

PBMC isolation and flow cytometry. PBMCs from RRMS subjects were isolated by centrifugation through Ficoll-Paque Plus (GE Life Sciences). PBMCs were frozen in 5% BSA and 10% DMSO before being thawed in a 37° C. water bath. Cells were then washed with 1% FCS in PBS and stained with 10% human serum to block FcRs before incubation with the following antihuman antibodies (Abs): FITC anti-CD24 (BioLegend), allophycocyanin anti-IgM (BioLegend), PerCP-Cy5.5 anti-CD19 (BioLegend), PE-Cy7 anti-CD27 (BioLegend), BV421 anti-IgD (BioLegend) and BV711 anti-CD38 (BioLegend). PBMCs were analyzed using an LSRII.

Transitional B cells were CD19⁺CD38^{hi}CD24^{hi}, naïve B cells were D19⁺CD24^{int}CD38^{int}CD27, CS-memory B cells were CD19+CD27+IgM-IgD-and plasmablasts were CD19⁺CD27⁺CD38^{hi}. Serum NFL and IL-1β RRMS patients. NFL and IL-1β from sera of patients were assessed by proximity extension assay (Olink Bioscience, Sweden) using the inflammation panel. Briefly, the assay uses oligonucleotide-labeled antibody pairs allowing for pair-wise binding to target proteins.

When antibody pairs bind target antigens, corresponding oligonucleotides form an amplicon, allowing for quantification of protein expression by high-throughput real-time PCR. Data is presented as normalized protein expression (NPX) values, Olink Proteomics' arbitrary unit on a log2 scale.

Statistical analysis. Data are presented as means±s.e.m. and statistical significance was determined using ANOVA. Differences were considered significant for P<0.05. Pearson correlation coefficient tests were used to measure linear correlations. Statistical analyses were performed using Prism 6 (GraphPad).

The inventors compared four different B cell subsets in the peripheral blood of MS patients either undergoing an active relapse, had recently recovered from a relapse or had stable disease activity for over one year (long-term stable) (Table 1).

TABLE 1
Random forest analysis comparing NFL only and the combination
of NFL, IL-1β and transitional B cells was performed.

| | | | | | |
|---|---|---|---|---|---|
| | | Recent | | | % |
| from\to | Active | recovery | Stable | Total | correct |

NFL

| | | | | | |
|---|---|---|---|---|---|
| Active | 5 | 3 | 1 | 9 | 55.556 |
| Recent recovery | 2 | 0 | 1 | 3 | 0.000 |
| Stable | 1 | 2 | 5 | 8 | 62.500 |
| Total | 8 | 5 | 7 | 20 | 50.000 |

NFL + IL-1β + Transitional B cells

| | | | | | |
|---|---|---|---|---|---|
| Active | 8 | 0 | 0 | 8 | 100.000 |
| Recent recovery | 0 | 3 | 3 | 6 | 50.000 |
| Stable | 0 | 2 | 4 | 6 | 66.667 |
| Total | 8 | 5 | 7 | 20 | 75.000 |

It was found that the percentage of transitional B cells was lowest in active relapse MS patients, intermediate in recently recovered patients and were highest in long-term stable MS patients (FIG. 1A-1B). In contrast, no difference was seen with naïve B cells, CS-memory B cells and plasmablasts in all three groups of MS patients (FIG. 1A). The inventors also examined the levels of the neuronal marker, NFL and the inflammatory marker, IL-1β in the sera of all three MS subgroups. Similar to previous reports, the inventors found that NFL levels were significantly elevated in active relapse MS patients (FIG. 1C). Strikingly, the inventors found a negative correlation between transitional B cells and NFL in this MS cohort (FIG. 1D). In addition, IL-1β levels were high in active relapse MS patients and recently recovered patients compared to long-term stable MS patients (FIG. 1E) and a negative correlation was also found between transitional B cells and IL-1β (FIG. 1F).

Next, random forest machine learning was used to compare the ability of NFL only or the combination of NFL, IL-1β and transitional B cells to predict MS relapse. It was found that the combination of NFL, IL-1β and transitional B cells was much better at predicting MS relapse than NFL alone.

The pursuit of finding specific biomarkers to improve clinical decision making and therapeutic approach is a hot topic of research in MS. The success of B cell depleting therapies reveals a critical role of B cells in driving neuro-inflammation. Regulatory functions of transitional B cells are essential for the efficacy of B cell modifying therapies in MS. Lack of reliable and specific diagnostic approaches at early stages of disease progression is a major hurdle in choosing optimal treatments for MS patients. Recent studies suggest that NFL levels in the blood of MS patients are associated with neuronal damage and disease activity, demonstrating the prognostic potential of NFL. However, using NFL as a biomarker for MS has been a subject of debate as NFL is considered a marker of neuronal damage in several neurological diseases, including MS. This study shows how combining transitional B cells along with the neuronal marker, NFL and the inflammatory marker, IL-1β can be used to assess disease activity and progression in MS patients.

In conclusion, this study demonstrates that transitional B cells along with serum NFL and IL-1β levels can be used in a biomarker panel to specifically predict MS relapses. This approach improves treatment decision making and therefore result in better therapeutic outcome in MS patients.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A method of treating a patient with multiple sclerosis (MS), the method comprising:
obtaining a hematopoietic cell sample from a patient having MS, wherein the patient was in relapse for MS;
determining the number of CD19⁺, CD24⁺, CD38⁺ transitional B cells in the hematopoietic cell sample, and a level of expression of neurofilament light (NFL) and an inflammatory marker; and
treating the MS patient with a therapeutic agent selected from at least one of: monomethyl fumarate, ozanimod, diroximel fumarate, cladribine, siponimod, ocrelizumab, daclizumab, alemtuzumab, PEG-interferon beta-1a, dimethyl fumarate, teriflunomide, fingolimod, natalizumab, laquinimod, ublituximab, or ponesimod, when the patient has a decreased number of CD19⁺, CD24⁺, CD38⁺ transitional B cells, and an increase in NFL inflammatory marker relative to an untreated MS control sample, an unresponsive MS control sample, or an long-term stable disease sample.

2. The method of claim 1, wherein the inflammatory marker is interleukin-1β (IL-1β).

3. The method of claim 1, wherein the determining step for CD19⁺, CD24⁺, CD38⁺ transitional B cells comprises:
contacting the hematopoietic cell sample with reagents that detect CD19, CD24 and CD38 as transitional B cell markers; and
quantitating those cells that are positive for expression of at least one of CD19, CD24 and CD38, when compared to a normal sample, or a sample from the untreated MS control sample, the unresponsive MS control sample, or the long-term stable disease sample.

4. The method of claim 3, wherein the determining step is performed by flow cytometry.

5. The method of claim 1, wherein the patient is human.

6. A method of treating a multiple sclerosis patient, the method comprising:
treating the MS patient with a therapeutic agent;
obtaining a hematopoietic cell sample from the patient;
determining the number of CD19⁺, CD24⁺, CD38⁺ transitional B cells in the hematopoietic cell sample, and a level of expression of neurofilament light (NFL) and interleukin-1β (IL-1β); and
continuing treatment of the MS patient with the therapeutic agent for MS when the patient has a decreased number of CD19⁺, CD24⁺, CD38⁺ transitional B cells and an increase in the expression of NFL and IL-1β relative to an untreated or unresponsive MS control sample or a patient with long-term stable MS, wherein the patient is determined to be responsive to the therapeutic agent.

7. The method of claim 6, wherein the determining step for CD19⁺, CD24⁺, CD38⁺ transitional B cells comprises:
contacting the hematopoietic cell sample with reagents that detect CD19, CD24 and CD38 as transitional B cell markers; and
quantitating those cells that are positive for expression of at least one of CD19, CD24 and CD38, when compared to a normal sample, or a sample from the untreated MS control sample, the unresponsive MS control sample, or the long-term stable disease sample.

8. The method of claim 7, wherein the determining step is performed by flow cytometry.

9. The method of claim 6, wherein the patient is human.

10. The method of claim 6, wherein the therapeutic agent is selected from at least one of: monomethyl fumarate, ozanimod, diroximel fumarate, cladribine, siponimod, ocrelizumab, daclizumab, alemtuzumab, PEG-interferon beta-1a, dimethyl fumarate, teriflunomide, fingolimod, natalizumab, laquinimod, ublituximab, or ponesimod.

11. A method of predicting and treating a recurrence of MS treating a patient with multiple sclerosis, the method comprising:
obtaining a hematopoietic cell sample from a patient suspected of having a recurrence of multiple sclerosis (MS), wherein the patient was in relapse for MS;
determining the number of CD19⁺, CD24⁺, CD38⁺ transitional B cells in the hematopoietic cell sample, and a level of expression of neurofilament light (NFL) and interleukin-1β (IL-1β), which is predictive of a recurrence of MS;
treating the MS patient with a therapeutic agent selected from at least one of: monomethyl fumarate, ozanimod, diroximel fumarate, cladribine, siponimod, ocrelizumab, daclizumab, alemtuzumab, PEG-interferon beta-1a, dimethyl fumarate, teriflunomide, fingolimod, natalizumab, laquinimod, ublituximab, or ponesimod when the patient has a decreased number of CD19⁺, CD24⁺, CD38⁺ transitional B cells, and an increase in NFL and inflammatory marker relative to the untreated MS control sample, the unresponsive MS control sample, or the long-term stable disease sample.

12. The method of claim 11, wherein the determining step for CD19⁺, CD24⁺, CD38⁺ transitional B cells comprises:
contacting the hematopoietic cell sample with reagents that detect CD19, CD24 and CD38 as transitional B cell markers; and
quantitating those cells that are positive for expression of at least one of CD19, CD24 and CD38, when compared to a normal sample, or a sample from the untreated MS control sample, the unresponsive MS control sample, or the long-term stable disease sample.

13. The method of claim 11, wherein the CD19⁺, CD24⁺, CD38⁺ cells are detected by flow cytometry.

14. The method of claim 11, wherein the patient is human.

15. A method of predicting a recurrence of MS treating a patient with multiple sclerosis, the method comprising:
obtaining a hematopoietic cell sample from a patient suspected of having a recurrence of multiple sclerosis (MS), wherein the patient was in relapse for MS; and
determining the number of CD19⁺, CD24⁺, CD38⁺ transitional B cells in the hematopoietic cell sample, and a level of expression of neurofilament light (NFL) and interleukin-1β (IL-1β), which is predictive of a recurrence of MS;
wherein an increase in CD19⁺, CD24⁺, CD38⁺ transitional B cells and/or a decrease in a level of expression of NFL and interleukin-1β (IL-1β) when compared to an untreated MS control sample, an unresponsive MS control sample, or an MS patient with long-term stable disease sample is indicative of a recurrence of MS.

16. The method of claim 15, wherein the individual is undergoing treatment with an at least one of: monomethyl fumarate, ozanimod, diroximel fumarate, cladribine, siponimod, ocrelizumab, daclizumab, alemtuzumab, PEG-interferon beta-1a, dimethyl fumarate, teriflunomide, fingolimod, natalizumab, laquinimod, ublituximab, or ponesimod.

17. The method of claim 15, wherein the determining step for CD19⁺, CD24⁺, CD38⁺ transitional B cells comprises:
contacting the hematopoietic cell sample with reagents that detect CD19, CD24 and CD38 as transitional B cell markers; and
quantitating those cells that are positive for expression of at least one of CD19, CD24 and CD38, when compared to a normal sample, or a sample from the untreated MS control sample, the unresponsive MS control sample, or the long-term stable disease sample.

18. The method of claim 15, wherein the CD19⁺, CD24⁺, CD38⁺ cells are detected by flow cytometry.

19. The method of claim 15, wherein the patient is human.

20. The method of claim 15, further comprising the step of treating the patient with at least one of: monomethyl fumarate, ozanimod, diroximel fumarate, cladribine, siponimod, ocrelizumab, daclizumab, alemtuzumab, PEG-interferon beta-1a, dimethyl fumarate, teriflunomide, fingolimod, natalizumab, laquinimod, ublituximab, or ponesimod.
